(19) United States
(12) Patent Application Publication
(10) Pub. No.: US 2024/0150279 A1
(43) Pub. Date: May 9, 2024

(54) C PRIME AGENTS FOR TREATING METABOLIC DISORDERS

(71) Applicant: The University of Toledo, Toledo, OH (US)

(72) Inventors: Paul Erhardt, Toledo, OH (US); Shi-He Liu, Toledo, OH (US); Francis Charles Brunicardi, Toledo, OH (US); Divya Andy, Toledo, OH (US)

(73) Assignee: The University of Toledo, Toledo, OH (US)

(21) Appl. No.: 18/274,549

(22) PCT Filed: Feb. 1, 2022
(86) PCT No.: PCT/US2022/014658

§ 371 Date: Jul. 27, 2023

Related U.S. Application Data
(60) Provisional application No. 63/144,161, filed on Feb. 1, 2021.
(60) Provisional application No. 63/208,807, filed on Jun. 9, 2021.

(51) Int. Cl. C07C 279/26; A61K 31/215; A61K 31/366; A61K 45/06; A61P 35/00; C07D 309/12

(52) U.S. Cl. CPC C07C 279/26; A61K 31/215; A61K 31/366; A61K 45/06; A61P 35/00; C07D 309/12

(57) ABSTRACT

Stably consolidated compounds assembled from a statin and a metformin analog, and metabolically labile dual prodrugs assembled from the same, methods of making both, and methods of using them to treat metabolic diseases such as cancers are described.

### RELATED APPLICATIONS

This application claims priority to U.S. Ser. No. 63/144,161 filed Feb. 1, 2021, and U.S. Ser. No. 63/208,807 filed Jun. 9, 2021, the disclosures of which are incorporated herein by reference in their entirety.

### STATEMENT REGARDING FEDERALLY SPONSORED RESEARCH

This invention was made with no government support. The government has no rights in this invention.

### BACKGROUND

Pancreatic cancer has a low survival rate that averages only 5 years after diagnosis. It is responsible for tens of thousands of deaths each year in the United States where pancreatic ductal adenocarcinoma (PDAC), in particular, is the fourth leading cause of cancer mortality. Effective drug treatments for pancreatic cancer can help to address this long-standing, unmet medical need worldwide.

Metabolic syndrome and related disorders are a growing problem across the globe, particularly in industrially advanced nations. Accordingly, there is a need for new and improved treatment options to help abate this new trend worldwide.

### SUMMARY

Provided are compositions comprising Formulas A, B, C and D as specified below.

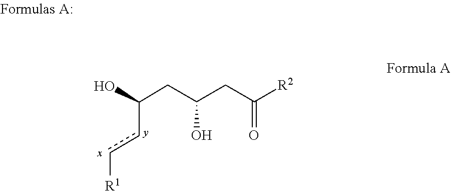

wherein R¹ is one of the following statin multi-cyclic core moieties R^{1a-f}

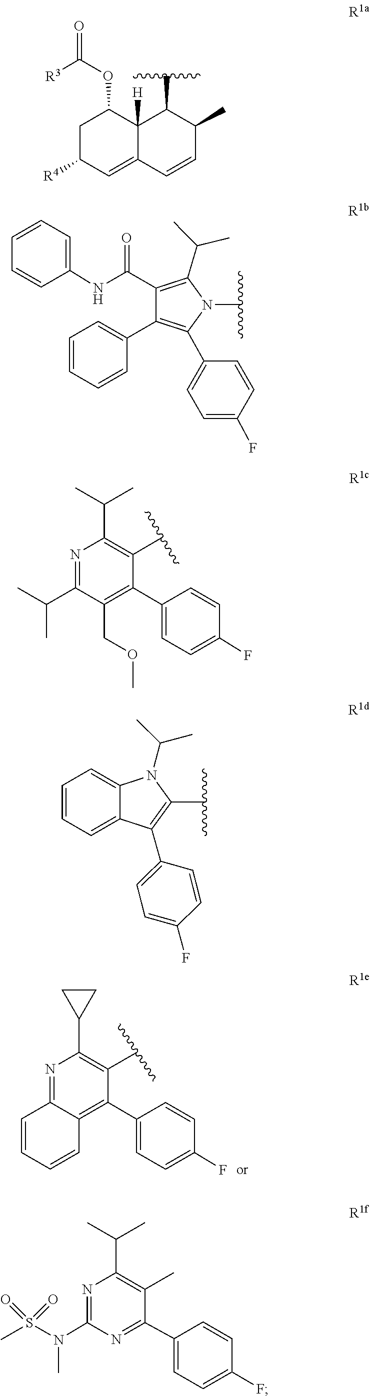

the x-y bond may be single or double having an (E)-conformation;
R² is

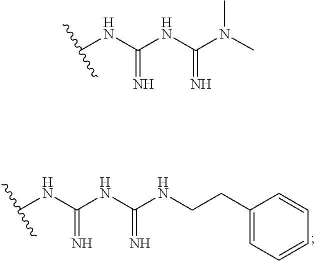

R³ is CH₂CH₂CH₃, CH(CH₃)CH₂CH₃ or C(CH₃)₂CH₂CH₃;
R⁴ is H, CH₃, OCH₃ or OH; and
said compounds include all enantiomer, racemic and diastereomeric possibilities, as well as simple hydrated and solvated physical forms.

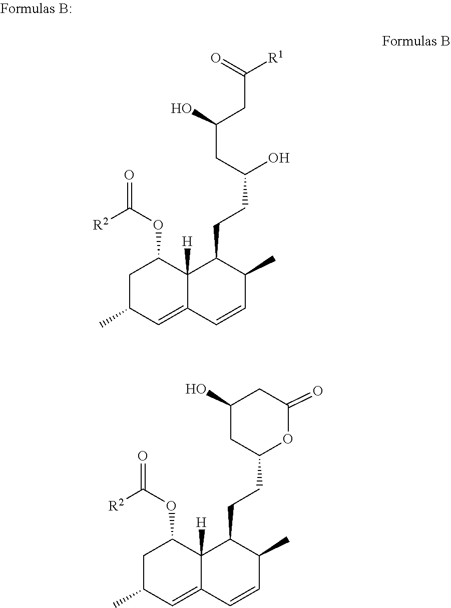

wherein R¹ is OH, OMe, OEt or NHOH;
R² is

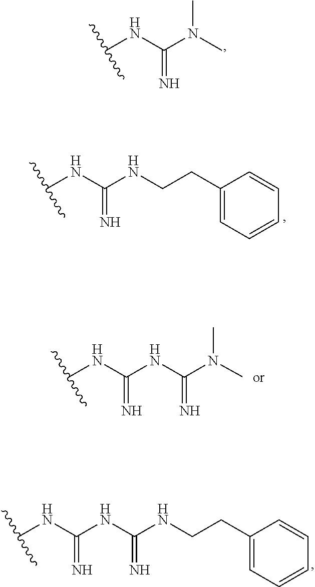

said compounds include all enantiomer, racemic and diastereomeric possibilities, as well as simple hydrated and solvated physical forms.

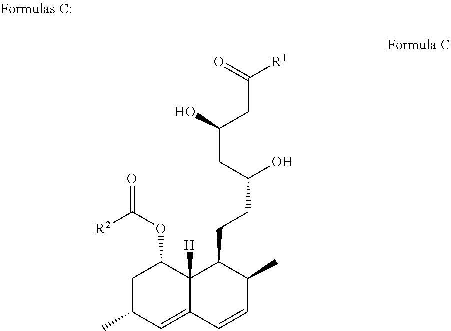

wherein R¹ and R² are together or independently

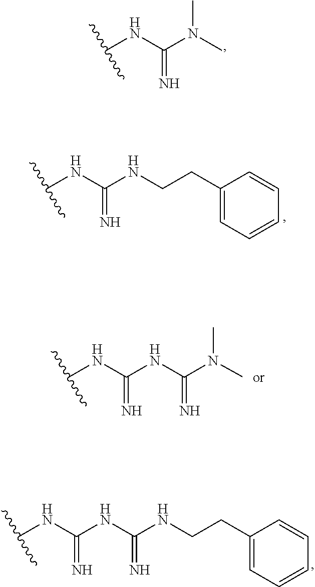

and said compounds include all enantiomer, racemic and diastereomeric possibilities, as well as simple hydrated and solvated physical forms.

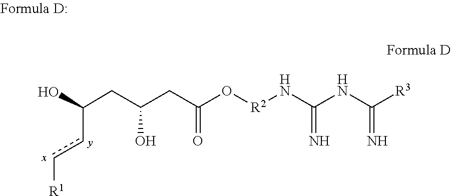

wherein R¹ is one of the following statin multi-cyclic core moieties R^{1a-f}

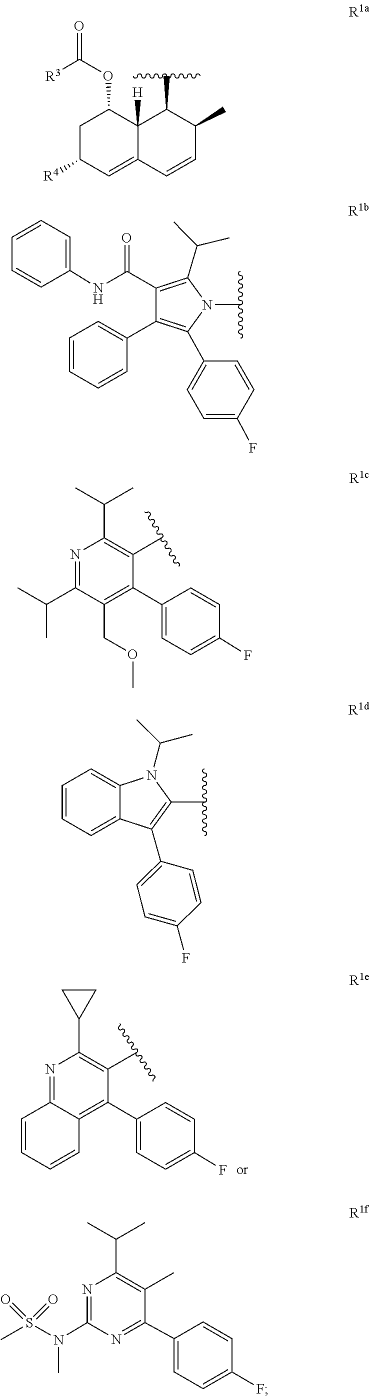

the x-y bond may be single or double having an (E)-conformation;
R² is CH₂, CH(CH₃), CH(CH₂CH₃), CH(CH₂CH₂CH₃), CH[CH(CH₃)₂], C(CH₃)₂, C(CH₂CH₃)₂, CH₂O(CO), CH(CH₃)O(CO), CH(CH₂CH₃)O(CO), CH(CH₂CH₂CH₃)O(CO), CH[CH(CH₃)₂]O(CO), C(CH₃)₂O(CO), C(CH₂CH₃)₂O(CO), CH₂CH₂C(CH₃)₂S, or

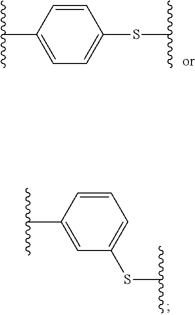 [SMILES: *Sc1ccc(*)cc1 ; *Sc1cccc(*)c1]

R³ is:

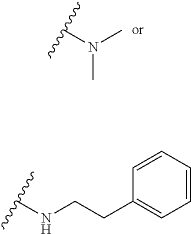

R⁴ is CH₂CH₂CH₃, CH(CH₃)CH₂CH₃ or C(CH₃)2CH₂CH₃;
R⁵ is H, CH₃, OCH₃ or OH; and
said compounds include all enantiomer, racemic and diastereomeric possibilities, as well as simple hydrated and solvated physical forms.

Further provided is a method of making a compound, the method comprising dissolving a statin and a biguanide in a first solvent and allowing the statin and biguanide to react to form a residue, and dissolving the residue in a second solvent to remove excess statin and produce a compound. In certain embodiments, the statin comprises simvastatin or lovastatin. In certain embodiments, the biguanide comprises metformin or phenformin. In certain embodiments, the first solvent comprises tetrahydrofuran (THF). In certain embodiments, the second solvent comprises dichloromethane (DCM).

Further provided is a method of attenuating the BIRC5-Survivin axis in order to treat certain cancers and to potentially treat certain metabolic disorders such as diabetes and hyperlipidemia, or a cardiovascular abnormality, the method comprising administering an effective amount of a compound of Formulas A, B, C or D to a subject. In certain composition of matter embodiments, the compound of formula A is administered wherein the R¹ group is R^{1a}, the x-y bond is a single bond, R³ is CH(CH₃)CH₂CH₃ or C(CH₃)₂CH₂CH₃, and R⁴ is CH₃.

In certain embodiments, the method further comprises administering a cardiac glycoside with the compound of formulas A, B, C or D. In particular embodiments, the cardiac glycoside comprises digitalis or digoxin. The co-administration of digoxin or a close analog is based upon a patient's individualized response to this specific type of multiple-drug therapy as assessed clinically, or for the case of cancer by either clinical response or ex vivo testing of tumor biopsy samples. In certain embodiments, treating cancer may additionally involve the co-administration of one or more well-established chemotherapeutic agents that rely upon prompting apoptosis to kill cancer cells. In certain embodiments, the cancer involves the pancreas or the latter's further sites of metastases.

In certain embodiments, the metabolic disorder resides in one or more of the following categories: glucose metabolism disorders, hyperlactatemia, lipid metabolism disorders, and phosphorous metabolism disorders. In certain embodiments, the metabolic disorder is associated with type 2 diabetes. In certain embodiments, the metabolic disorder is associated with hyperlipidemia. In certain embodiments, the metabolic disorder is associated with metabolic syndrome.

Formulas A, B (with R¹=OH or NHOH) and C are stable chemical constructs designed to behave like a single drug molecule that possesses multiple beneficial effects within the body. Formula B with R¹=OMe or OEt, is a simple prodrug form that rapidly collapses to R═OH in the body by the action of ubiquitous esterases. A method for similarly treating cancer or metabolic disorders is also provided herein by alternatively administering a dual prodrug comprising a metformin or phenformin analog connected to a statin analog using metabolically labile chemical bonds such that the connection intentionally becomes severed in vivo after administration to humans. Both metabolites then simultaneously display their beneficial effects within the body. While not intending to limit the scope for such dual prodrug possibilities, Formula D is representative of our novel metabolically labile connections.

### BRIEF DESCRIPTION OF THE DRAWINGS

Copies of this patent or patent application publication with color drawings will be provided by the U.S. Patent and Trademark Office upon request and payment of the necessary fees.

FIG. 1: Depiction of the PDX1-BIRC5-Survivin axis showing the pathway that can lead directly to apoptosis in cancer cells when it is inhibited by drugs. Also shown are paths associated with ‘various protein interactions and signaling pathways’ that can impact upon cellular metabolic processes. The dashed-line feedback loops may have either inhibitory or enhancing properties depending upon which protein interactions and signaling pathways become involved. Homeostasis reflects a balance among all pathways in a continuous dialogue that also attempts to address cellular insults and stress.

FIG. 2: Structures of metformin (“Met”) and simvastatin (“Sim”), along with the free alcohol form of Sim, the opened lactone of Sim, and the free alcohol form of Sim's opened lactone.

FIG. 3: Chemical structures of Sim and Met, of metabolically labile dual prodrug compounds using esters and thio-amine linkers shown in green, and of metabolically stable compounds that use either sterically hindered amide linkages at the satin 1-position or sterically hindered carbamate linkages at the statin 2-position, both shown in red and using numbering taken from Sim's structure.

FIG. 4: Hydroxamic acid analog of simvastatin.

FIG. 5: Scheme depicting the synthesis of SAR probes.

FIG. 6: Scheme depicting the synthesis of consolidated combination compounds at the statins' lactone site.

FIG. 7: Scheme depicting the synthesis of Sim−Met and Lov−Met at the statins' ester site.

FIG. 8: Scheme depicting the synthesis of a statin consolidated with two Mets.

FIG. 9: Schemes depicting the synthesis of exemplary dual prodrugs.

FIG. 10: Biological activity of consolidated Met−Sim (aka Met/Sim or MS) plus digoxin (Dig) compared to simultaneous administration of Met, Sim, and Dig (independent compounds mixture aka C3).

FIG. 11: Biological activity of Met−Sim compared to simultaneous administration of Met and Sim.

FIG. 12: Graph showing the effects of Met−Sim (“MS”) or Met plus Sim individually on ATP levels in rapidly dividing cancer cells. Rapidly dividing cancer cells (e.g., “PDCL5”) tend to favor energy metabolism by glycolysis rather than using the oxidative phosphorylation pathway (Warburg effect). Drug treatment with either a mixture of metformin and simvastatin (“Met+Sim”) or by our novel construction of a single Met−Sim (“MS”) molecule disturbs this energy supply (shown in FIG. 12 as decreasing levels of ATP) which, in turn, leads to cancer cell death by starvation rather than apoptosis.

FIG. 13: Graph showing the effects of our novel Met−SIM (“MS”) single molecule construct versus Met plus Sim individually on ATP levels in Mia PaCa2 cancer cells.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Provided herein are compounds that target BIRC5 for therapy of pancreatic ductal adenocarcinoma (PDAC), among other diseases. BIRC5 is a biomarker and therapeutic target of PDAC. The compounds described herein are either single molecule drug constructs having pleotropic effects or are dual prodrugs that release two single drug molecules that simultaneously display their own effects. Both types inhibit cell proliferation of PDAC cells in vitro and in vivo via suppression of BIRC5 expression. The compounds have enhanced anti-tumor effects by targeting BIRC5.

In some embodiments, the compounds herein have Formula A, B, C, or D as shown below.

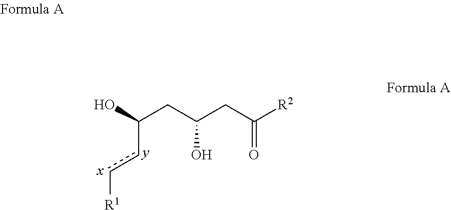

wherein R¹ is one of the following statin multi-cyclic core moieties R^{1a-f}

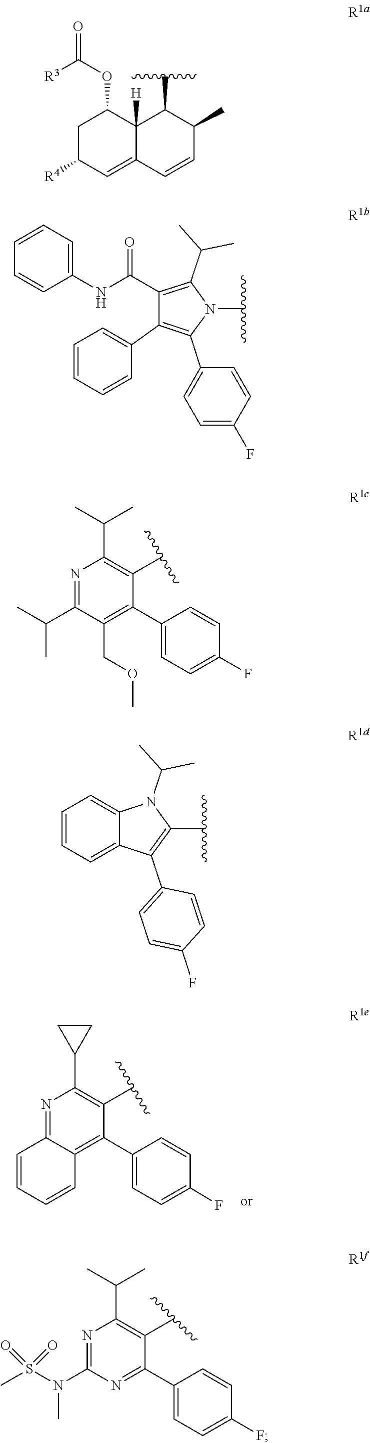

the x-y bond may be single or double having an (E)-conformation;
R² is

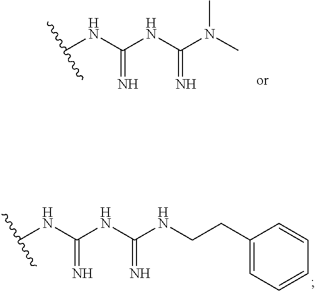

R³ is CH₂CH₂CH₃, CH(CH₃)CH₂CH₃ or C(CH₃)₂CH₂CH₃;
R⁴ is H, CH₃, OCH₃ or OH; and
said compounds include all enantiomer, racemic and diastereomeric possibilities, as well as simple hydrated and solvated physical forms.

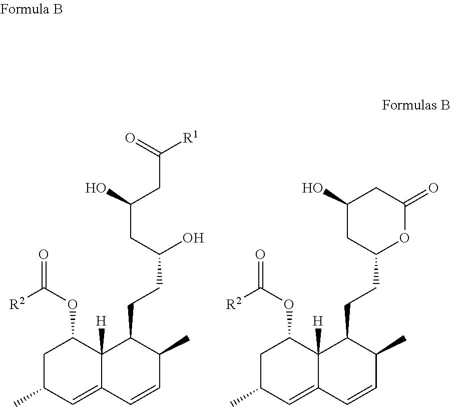

wherein R¹ is OH, OMe, OEt or NHOH;
R² is

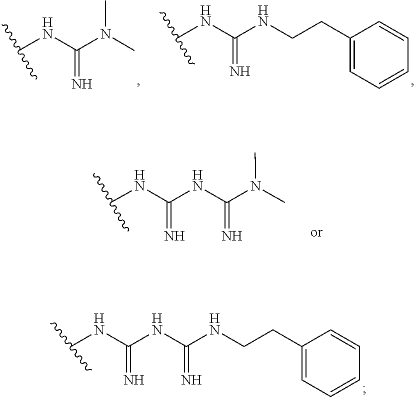

and
said compounds include all enantiomer, racemic and diastereomeric possibilities, as well as simple hydrated and solvated physical forms.

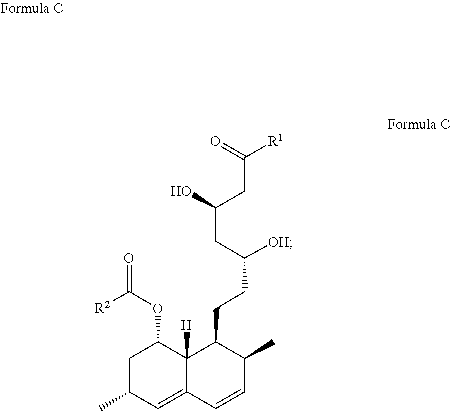

wherein R¹ and R² are together or independently

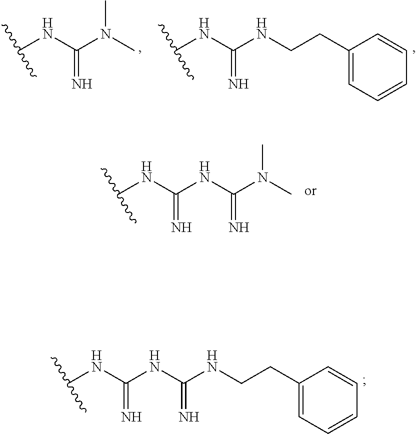

said compounds include all enantiomer, racemic and diastereomeric possibilities, as well as simple hydrated and solvated physical forms.

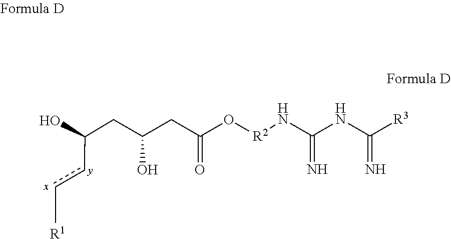

wherein R¹ is one of the following statin multi-cyclic core moieties R^{1a-f}

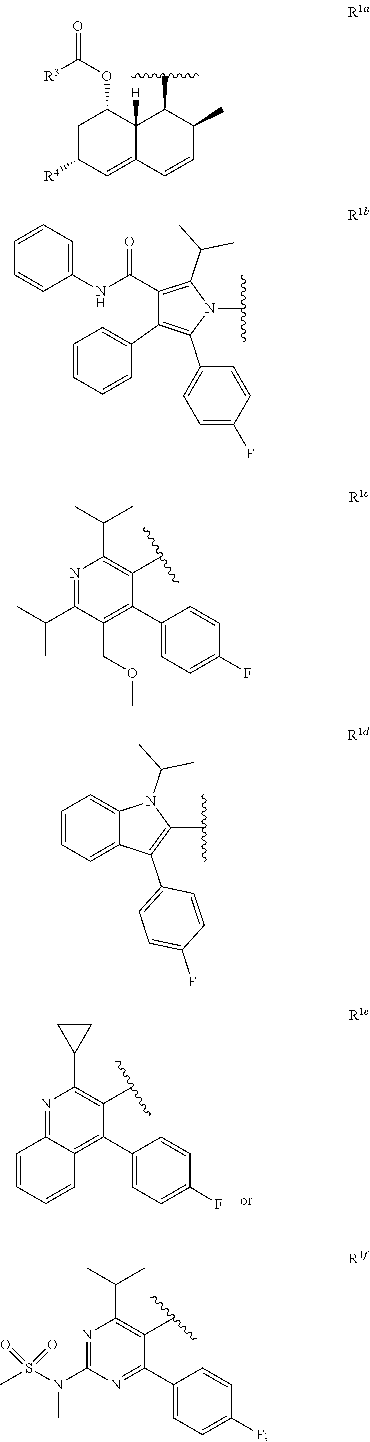

the x-y bond may be single or double having an (E)-conformation;
R² is CH₂, CH(CH₃), CH(CH₂CH₃), CH(CH₂CH₂CH₃), CH[CH(CH₃)2], C(CH₃)₂, C(CH₂CH₃)₂, CH₂O(CO), CH(CH₃)O(CO), CH(CH₂CH₃)O(CO), CH(CH₂CH₂CH₃)O(CO), CH[CH(CH₃)_{2]}O(CO), C(CH₃)₂O(CO), C(CH₂CH₃)₂O(CO), CH₂CH₂C(CH₃)₂S, or

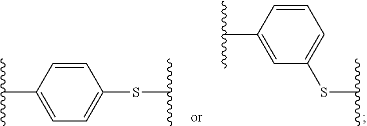 [SMILES: *Sc1ccc(*)cc1 ; *Sc1cccc(*)c1]

R³ is

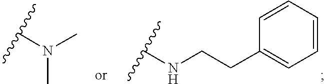

R⁴ is CH₂CH₂CH₃, CH(CH₃)CH₂CH₃ or C(CH₃)₂CH₂CH₃;
R⁵ is H, CH₃, OCH₃ or OH; and
   said compounds include all enantiomer, racemic and diastereomeric possibilities, as well as simple hydrated and solvated physical forms.

Our nomenclature for the single molecular constructs attempts to track both the statin component and the biguanide component. For example, within Formula A when R¹ is R^{1a} with R³ being C(CH₃)₂CH₂CH₃ and R⁴ being CH₃, and when x-y is a single bond and R² is NHC(NH)NHC(NH)NMe₂, the compound is known as ‘Met−Sim’ (also referred to as ‘Met/Sim’ or ‘MS’ or ‘Sim−Met’ or ‘Sim/Met’ or ‘SM’) because the components of our novel single compound are a chemically stable combination of metformin and simvastatin. Alternatively, within Formula A when R¹ is R^{1a} with R³ being CH(CH₃)CH₂CH₃ and R⁴ being CH₃, and when x-y is a single bond and R² is NHC(NH)NHC(NH)NMe₂, the consolidated compound is known as ‘Met−Lov’ (also referred to as ‘Met/Lov’ or ‘ML’ or ‘Lov−Met’ or ‘Lov/Met’ or ‘LM’) because its components are metformin and lovastatin combined as a novel single molecule. As shown in the examples herein, both Sim−Met and Lov−Met inhibit growth of pancreatic cancer cells. This has been demonstrated using multiple pancreatic cancer cell lines including some taken directly from cancer patients as well as from established cell lines. Further, we have shown that this beneficial activity is associated with both enhancement of the PDX1-BIRC5-Survivin axis's normal metabolic functions and attenuation of its abnormal production of survivin during cancer. Likewise, we have demonstrated that there is a favorable impact upon ATP levels, and in some patient's cell lines we have shown Met−Sim to be more effective at inhibiting pancreatic cell growth than the simultaneous administration of metformin and simvastatin as their individual drug forms.

The compounds of Formula D are novel prodrugs, also referred to herein as ‘dual prodrugs’. The dual prodrugs of Formula D have doubled the simpler and more common ester prodrug chemical motifs into a chemical format that can free-up two distinct drugs rather than just a single parent drug. The acidic nature of the stomach should protect esters from spontaneous hydrolysis, but even if this does occur, the liberated agents are themselves bioavailable.

The compounds of Formulas A, B and C are single molecule hybrid combinations which we also call ‘consolidated compounds.’ The compounds of Formula D are dual prodrug combinations and we refer to them as such. Both types of combinations involve analogs of metformin or phenformin connected to statin analogs that together can uniquely attenuate the PDX1-BIRC5-Survivin axis in a manner conducive to treating metabolic disorders such as certain cancers, diabetes, hyperlipidemia, metabolic syndrome, and certain cardiovascular abnormalities. The compounds of Formulas A, B, and C are referred to herein as consolidated compounds because they effectively unite the activity of metformin and phenformin analogs with the activity of statin analogs in single compounds that, in some cases, distinctly results in better activity than the simultaneous administration of metformin and a statin individually or in their formulated mixture combinations. Furthermore, in some embodiments, a cardiac glycoside such as digitalis or digoxin may be administered together with one or more compounds of Formulas A, B, C or D to enhance the overall therapeutic effect, particularly with regard to individualized treatments of cancer patients.

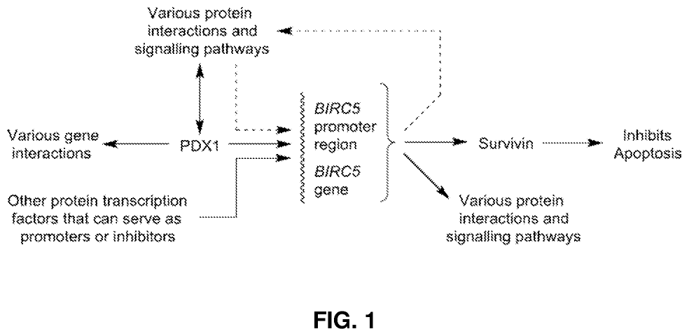

A key characteristic of several metabolic disorders involves aberrant functioning of Pancreatic and Duodenal Homeobox 1 (PDX1), a multi-pathway signaling protein and a transcription factor that regulates several genes involved in cellular development and homeostasis. Among this array of interactions, PDX1 activates the Baculoviral IAP Repeat Containing 5 (BIRC5) gene promoter. BIRC5, in turn, expresses the anti-apoptotic protein called ‘survivin.’ This relationship is depicted in FIG. 1.

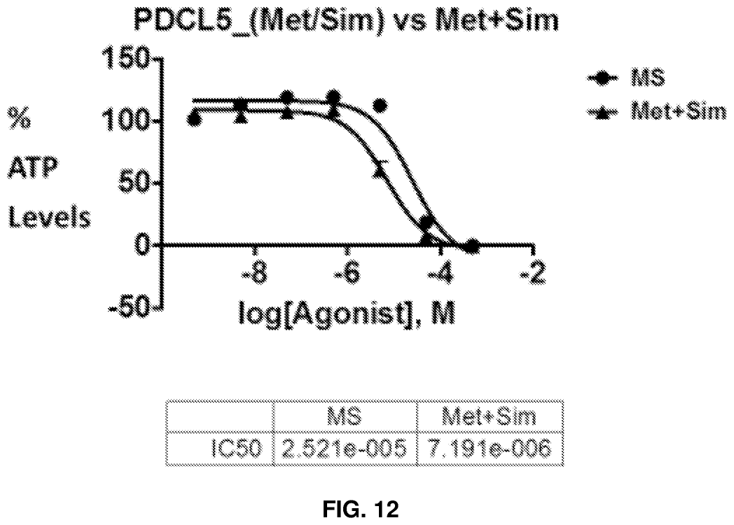

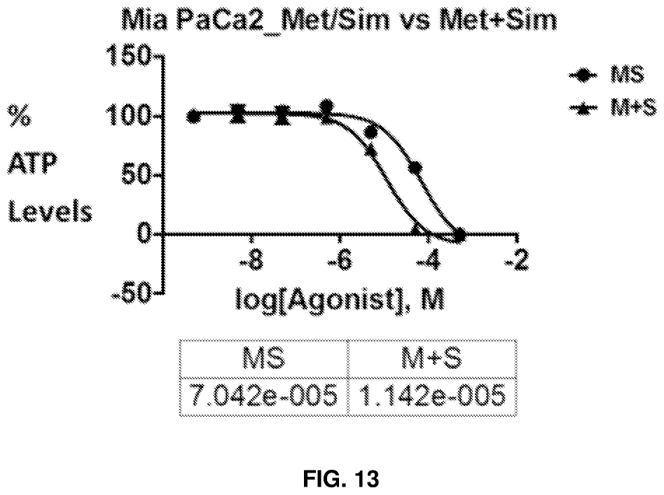

We have shown that drug-induced inhibition of the PDX1-BIRC5-Survivin axis can attenuate pancreatic ductal adenocarcinoma (PDAC). Deploying a novel triple formulated combination of metformin, simvastatin, and digoxin (aka C3) was found to decrease cell proliferation associated with inhibition of BIRC5 expression in PDAC cells, and to markedly suppress BIRC5 and the growth of PDCL tumors in mouse models. We recently reported these findings in Cancer Letters, 491 (2020) pages 97-101 (S-H. Liu et al.). In accordance with the present disclosure, inhibition of the PDX1-BIRC5-Survivin axis can also reduce aberrantly high metabolic activity in cancer cells when assessed by measuring ATP levels. (FIGS. 12 and 13). Metabolic disorders that may benefit from such treatment are type 2 diabetes and hyperlipidemia (e.g., high cholesterol), as well as cancer wherein its treatment thus benefits from a composite of activities including attenuation of the Warburg effect in addition to restoration of apoptosis. Restoration of metabolic balance, including cholesterol homeostasis, is particularly useful for the treatment of metabolic disorders.

There are currently no clinically used drugs that target the PDX1-BIRC5-Survivin axis as the mechanism responsible for their beneficial effects. There have been single agents that target BIRC5-Survivin, and our simultaneous deployment of three separate drugs (which we call ‘C3’) has been used to treat pancreatic cancer where the benefit is thought to derive from a combination of multiple interactions along the PDX1-BIRC5-Survivin pathway. However, there remains a complete lack of single molecules that target the PDX1-BIRC5-Survivin axis.

C3's three drugs are metformin, simvastatin, and digoxin. The administration of this formulated combination of three drugs to treat pancreatic cancer is described in United States Patent Application Publication 2019/0358193 A1 (F. C. Brunicardi and R. Sanchez), which is incorporated herein by reference. Previous research led to a super-promoter assay that can be used to identify small molecules able to disturb the BIRC5-Survivin pathway, where such compounds are potential anticancer agents. Several known compounds that are not normally considered chemotherapeutic agents were identified as being especially effective in this assay. Various combinations of these agents led to a specific mixture that had reasonable activity in a consistent manner across commercial and several patient-donated pancreatic cancer cell cultures. C3 is one such distinct combination. The consolidated and dual prodrug compounds described herein display the beneficial properties related to disturbing BIRC5 that are present in each of the C3 members but do so as a single compound or as a metabolically labile dual prodrug.

Metformin (“Met”) is the first-line medication for the treatment of type 2 diabetes, particularly in overweight patients. It decreases the liver's production of glucose, has an insulin-sensitizing effect on multiple tissues including adipose, and enhances peripheral glucose uptake. In addition to Met's connections with metabolic diseases like diabetes and hyperlipidemia, there are only partially understood pathways that appear to connect some of insulin's actions with PDX1 signaling and therein likely also with BIRC5 according to findings herein for this latter axis (FIG. 1). For example, PDX1 expression is required for maintenance of β-cells in the matured pancreas where, interestingly, low concentrations of insulin protect them from apoptosis except when PDX1 expression has been inhibited. There are conflicting results on overall survival when Met has accompanied clinical treatments of pancreatic cancer. In terms of metabolic effects, it has been shown that Met's interaction with the AMPK pathway decreases HMG-CoA reductase activity which, in turn, lowers elevated cholesterol levels in the endoplasmic reticulum.

To treat diabetes, Met has been used clinically in several two-drug formulated combinations and in a three-drug formulated combination, the stated intent of which is to reduce pill burden, simplify chronic administration, and improve patient compliance. However, none of these combinations include a statin drug, and all of them are formulated mixtures rather than being chemically merged compositions joined as either dual prodrugs or in a stable-bonded manner that maintains a single consolidated chemical species in vivo.

Met has been present with statins in clinical settings addressing cancer patients with additional illnesses. While the statins improved overall survival, metformin alone or in combination did not. In the antiviral arena, Met has been used in combination with either lovastatin or simvastatin, and in both instances, the combinations have been beneficial. However, these combinations were again only formulated mixtures, not dual prodrugs or chemically merged (consolidated) compositions.

We found that Met can be derivatized at one of its N atoms without losing activity in the PDX1-BIRC5-Survin assay such that a specific connection of this type can be utilized to create a consolidated compound. Met can also lend itself to analogous constructs for the dual prodrug type of compounds by utilizing the well-established oxymethylene insertion and self-immolative moiety, both of which spontaneously collapse after ester hydrolysis by metabolizing enzymes.

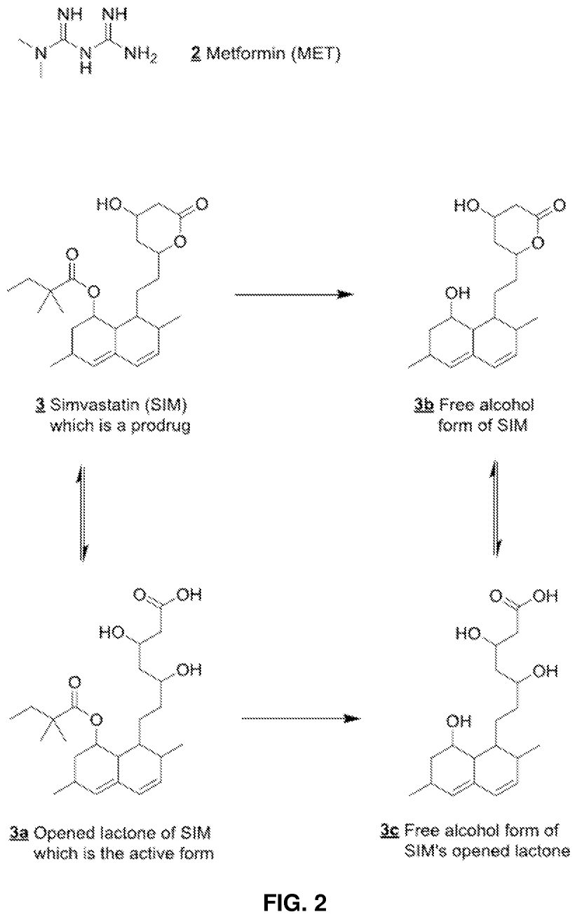

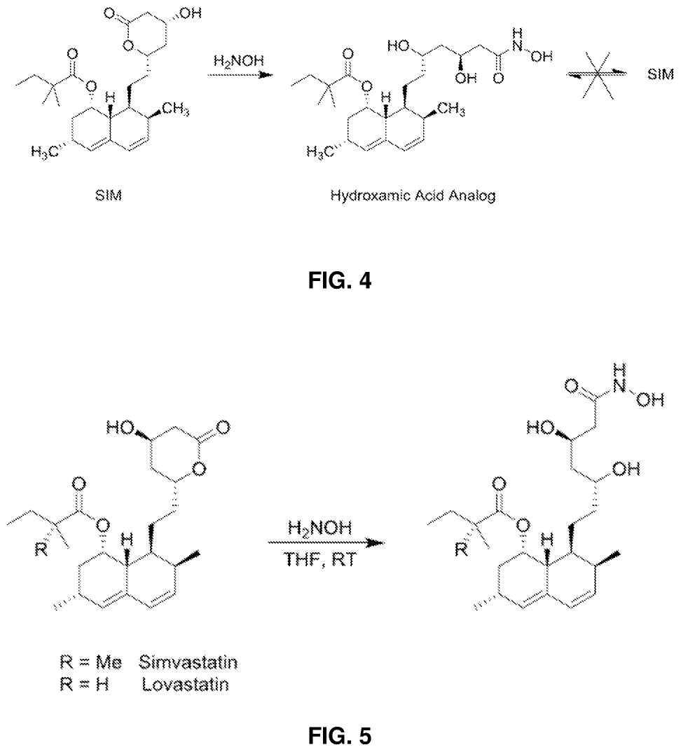

Statins are the most common medications for the treatment of high cholesterol and hyperlipidemia. They inhibit HMG-CoA reductase and thus decrease the liver's synthesis of cholesterol. Reduced levels of cholesterol, in turn, prompt cells to express higher numbers of LDL receptors to draw cholesterol out of the circulation. There are mixed views about the risk of developing diabetes as a side-effect, and concrete mechanistic connections for this possibility remain unclear. Cholesterol synthesis is known to be important for the production of proteins such as GLUT1 which is associated with cellular uptake of glucose, so when these are inhibited, there is a decrease in a cell's uptake of glucose in response to circulating insulin. However, there also appears to be additional pleotropic effects of the statins that are beneficial even if not well understood. Thus, by appropriately balancing the magnitude of interactions across various of these signaling pathways (with positive and negative feedback loops), therapeutic benefits may even be further gained by a distinct combination of agents or a chemically-combined-agent ideally tailored to accomplish this. Common statins include simvastatin (“Sim”), lovastatin (“Lov”), atorvastatin, fluvastatin, and pravastatin. Sim is actually a prodrug that relies upon the ring-opened version to bestow its lipid-lowering activity. The ring-closed form of Sim is inactive as an HMG-CoA reductase inhibitor while the form for its active role in C3 remains to be established. The lactone of simvastatin is in equilibrium with its open-ring form within the body, which allows for its use in either form as a prodrug/drug. These relationships are shown in FIG. 2. It is the open form that is active for its inhibition of HMG-CoA reductase. It is believed that the lactone and its opened ring version are a key functional group for activity. Hydroxamic acid analogs that model the acidic moiety present in the opened lactone of the statins effectively lock the compounds into this opened form. This is shown in FIG. 4 for Sim. Fluvastatin (“Flu”) is the ring-opened and active version of a statin where the close-ring lactone form serves as a prodrug. Either the lactone or ring-opened carboxylic acid version can be used.

Similar to Met, there are conflicting results on overall survival when statins have accompanied clinical treatments of pancreatic cancer, although the statins appear to be more consistently beneficial in general. Both the principal mechanism of action for the statins (i.e., inhibition of HMG-CoA) and one of its pleiotropic effects that impacts leukocyte function-associated antigen-1 (LAF-1) have been shown to be advantageous.

The open-closed-ring equilibria species for the statins has been well-studied, and the open form has been found to be active, a feature known to be requisite for inhibition of HMG-CoA reductase whether or not the latter is relevant to the BIRC5-related activity. We found and report herein that the resulting carboxylic acid's acidity can be attenuated to the level of a hydroxamic acid (—CONHOH) while retaining its activity in the BIRC5-survivin assay. Thus, this novel structure-activity relationship (SAR) suggests that a hydroxamic acid is a reasonable surrogate for Sim's opened-ring carboxylic acid moiety relative to interactions involving the BIRC5 axis. A —CONHOH group has been previously shown to be an acceptable bioisostere for a carboxylic acid group relative to the statins more classic HMG-CoA reductase activity. These findings were previously reported in the Journal of Medicinal Chemistry, 56 (2013) pages 3645-3655 by J.-B. Chen et al. In their regard, it can be further noted that a —CONHOH moiety is also a common feature in HDAC inhibitors because of its well-characterized Zn-chelating properties and not because it is generally accepted as a bioisostere for a carboxylic acid group.

Surprisingly, we additionally found that the requisite carboxylic acid of the statins is able to be mimicked by an amide made from combination with Met. Met is a biguanide, and guanidine is the most basic moiety in the body. The guanidine moiety is one of the strongest of the endogenous bases. From this chemical behavior, it is surprising that the Met-statin compounds are active in the way they are, as one would expect the opposite. However, because the guanidine is so strongly basic, it is essentially completely protonated at physiological pH. Thus, its protonated form is a weak conjugate acid. Our SAR studies noted above have revealed that when the statins' carboxylic acid group is substituted with a hydroxamic acid moiety, the desired activity in the biological assays for the PDX1-BIRC5-Survivin axis and cancer cell cultures is retained. Without wishing to be bound by theory, we believe that this particular stable chemical combination works because a protonated form of an acylated guanidine (e.g., acylated-MET or acylated-phenformin) becomes ideal for mimicking the lower acidic level that we demonstrated to be adequate in a statin (i.e., Sim-CONHOH) for it to be active in the specific PDX1-BIRC5-Survivin bioassays that are central to the instant invention.

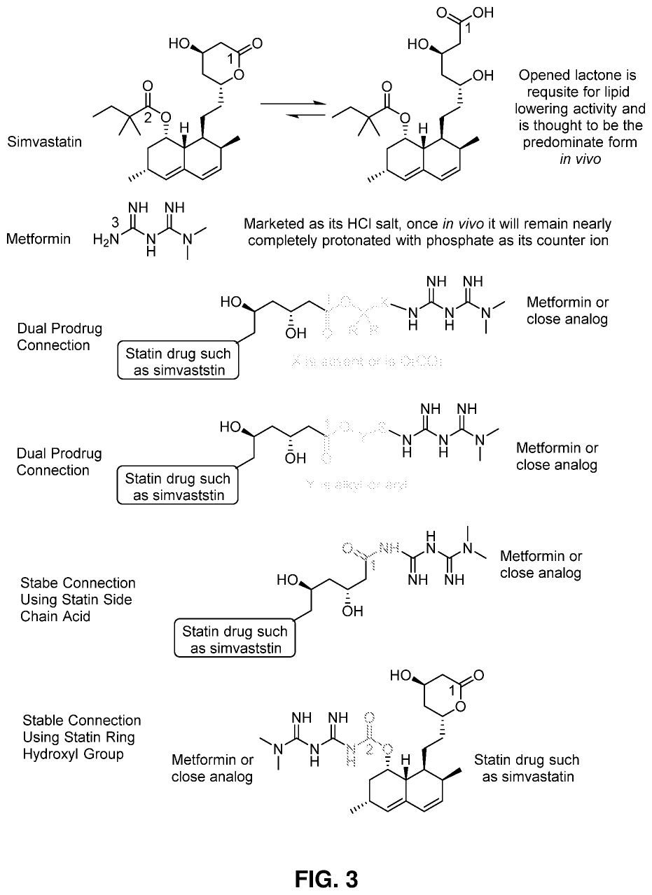

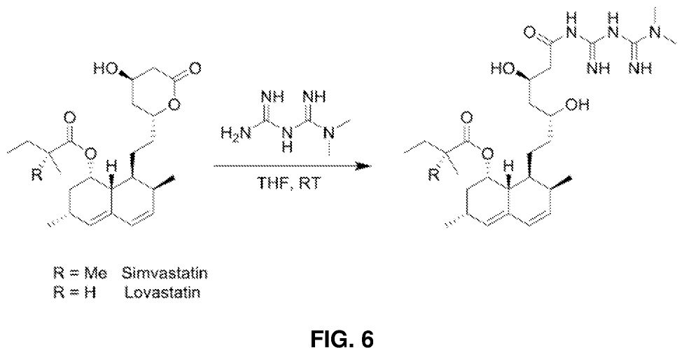

The compounds described herein combine the requisite structural features of statins like metformin and simvastatin, in either a dual prodrug arrangement or a metabolically stable, chemical bond-connection consolidated manner. Non-limiting examples of these combinations' chemical connections are depicted in FIG. 3. Notably, the steric hindrance additionally stabilizes the amide and carbamate linkages (red units at statin sites 1 or 2) when compared to the metabolically labile ester arrangements (green unit at statin site 1). Also, the metabolically stable connections can both be used within a single statin so as to produce a compound having a mole ratio of one statin to one Met, or a mole ratio of one statin to two Mets, as shown in FIGS. 6, 7 and 8.

Interestingly, our SAR may pertain not only to the mechanistic ‘black boxes’ associated with drug actions impinging upon the PDXI-BIRC5-Survivin axis, but also may be applicable to the known mechanism associated with the well-established actions of the statin drugs in their inhibition of HMG-CoA reductase. This is because the latter's catalytic domain amino acids that bind the statins' requisite carboxylic acid (or acid surrogates in tour compounds herein) have considerable flexibility so as to accommodate steric bulk present in its ligands. The Met-statin compounds, with very large surrogate acid moieties, may take advantage of this active site feature in a unique and distinctive manner if they try to bind in a similar fashion to HMG-CoA reductase. Alternatively, the mechanisms for Mets' well-established therapeutic actions are not fully understood. Enhancement of AMP-activated Protein Kinase (AMPK) has been implicated as a major mechanistic feature, possibly by activation of the c-Src/PI3K pathway. But wherever the location for the beneficial mechanistic interactions, the basicity of Met's guanidine is altered by its acylation during construction of the consolidated compound in which it is connected to a statin, and again, the resulting steric bulk of the resulting drug is significantly increased. Again, without wishing to be bound by theory, it is believed that the conformational flexibility afforded by the statin side-chain may either be able to (i) orient any problematic bulk in a direction away from an energetically favorable binding domain on proteins associated with Met's desirable actions, or (ii) allow for additionally favorable hydrophobic interactions in a near-by vicinity of such proteins' active sites. Some support for this proposal that our distinct BIRC5-related SAR might also apply to HMG-CoA reductase interactions, may be gleaned from the International Patent publication WO 2008/157537 A2 by M. Currie et al. who, among numerous other unrelated combinations, connected statins to guanidine and to aminoalkylguanidines for the purpose of treating lipid disorders. While the HMG-CoA reductase activity is not clearly defined, it is implied by the specified indication. Also note that in their case the guanidine adducts were intended to serve as soluble guanylate cyclase modulators/NOS substrates rather than as metformin or phenformin mimics. This key difference is also reflected in their preferred compositions being restricted to un-substituted guanidine or a single methyl-substituted guanidine whereas our preferred bis-guanidine compounds deploy either a dimethylguanidine-guanidine arrangement so as to be specifically analogous to metformin, or deploy a phenethylguanidine-guanidine arrangement so as to be specifically analogous to phenformin, as well as deploying metformin and phenformin themselves. Thus, our distinctively different combinations prompt the specific benefits associated with each of these well-known clinical agents as key aspects of our novel consolidated molecules and novel dual prodrug compounds.

The consolidated and dual prodrug compounds described herein are useful therapeutically and have practical advantages. The convenience of taking a single medication compared to two medications is a more user-friendly situation that can enhance a patient's/consumer's compliance with the prescribed protocol. Moreover, it has been found that the consolidated compounds herein are sometimes synergistic (compared to Met or Sim alone) in the overall benefit, as demonstrated at the PDX1-BIRC5-Survivin mechanistic level and in cell-based assays relative to anticancer activity. Our data suggests that the compounds may be useful for the treatment of hyperlipidemia, diabetes, metabolic syndrome, as well as to certain cancers such as cancers of the pancreas and bowel/GI tract.

The consolidated and dual prodrug compounds described herein may provide the benefits of a lessened pill burden, less cumbersome administration, improved patient convenience and compliance, reduced side-effects, enhanced safety, and longer maintenance of therapeutic concentration. Furthermore, there are distinct advantages that our compounds can additionally offer compared to formulated mixtures of, for example, metformin and simvastatin. For the consolidated and dual prodrug compounds, the entry of each component into the systemic circulation will indeed be simultaneous because factors affecting the amount and rate of absorption of the single entity after oral administration will necessarily affect both of the chemically-combined-components as a single chemical entity. This initial step is the absorption (‘A’) portion of the overall PK profile known as ‘ADME’ (absorption, distribution, metabolism, and excretion). To the contrary, all of the A factors will apply separately to each of the components within a formulated mixture upon its dissolution in the GI tract, a requisite initial step that formulations cannot avoid in order for absorption to occur. This type of distinction will continue to prevail as the single-species consolidated compound versus two separately administered agents move through the periphery to eventually arrive at their sites of action while simultaneously being subject to the body's attempts to degrade them by metabolism (the distribution and metabolism portions of ADME). Furthermore, the dual prodrugs of Formula D are able to fine-tune metabolic half-lives via steric hindrance by incorporating R groups of stipulated sizes at strategic molecular locations, and therefore can also benefit from this assured mutual delivery.

The ADM steps are important because the cell culture results shown in the examples herein have demonstrated that the agents being present together near the PDXI-BIRC5-Survivin axis across the same period of time, can indeed be synergistic compared to administering the single agents at staggered times and varying combinations.

The final ADME benefit pertains to excretion, E. As mentioned above, the ambiguous complexity of having so many singular agents competing across the same families of biological transporter systems where each can vary from individual to individual, becomes significantly lessoned when the agents are chemically combined so as to be present as just one composition. Drug-drug interactions that can detract from overall therapy will be completely avoided because there will be only one drug present rather than two. For similar reasons, the compounds described herein can allow for extensions in half-life (possibly once-per-day dosing) by exploiting the same design motifs described above. In contrast to our consolidated and dual prodrug compounds, delayed-release formulations of the individual agents remain complicated to control, especially when dealing with more than one active ingredient. Also, if depot formulations are administered, then some of the initial attributes mentioned above may no longer remain applicable for simple formulation approaches. Thus, the consolidated and dual prodrug compounds described herein are at least as effective, if not much better in terms of the ADME/PK profile, than formulated mixtures of metformin and simvastatin, and further provide for individualized therapy with fewer side effects.

Advantageously, the compounds described herein can be produced from synthetic routes that can begin with inexpensive starting materials and utilize common, environmentally friendly reagents and solvents. Only a few steps are needed, and many species can be assembled in just a single step from their starting materials. For example, a statin such as simvastatin and a guanidine derivative or biguanide such as metformin or phenformin, can be dissolved in a first solvent and allowed to react and form a residue, which can be dissolved in a second solvent to remove excess starting materials and directly produce a compound that can be further purified. Suitable solvents include, but are not limited to, tetrahydrofuran (THF) and dichloromethane (DCM). Non-limiting example schemes for making the consolidated compounds are shown in FIGS. 5-8. The dual prodrugs can be assembled from classical prodrug textbook methods such as those described or further referenced in Prodrugs: Strategic Deployment, Metabolic Considerations and Chemical Design Principles, P. Erhardt et al. in Burger's Medicinal Chemistry, Drug Discovery and Development, 7^{th} Edition. Edited by D. Abraham. John Wiley & Sons, Inc. Hoboken, New Jersey, 2010, pages 103-150, or in Prodrugs and Targeted Delivery, J. Rautio et al. in Methods and Principles in Medicinal Chemistry, Volume 47, Edited by R. Mannhold et al. Wiley-VCH Verlag & Co., Weinheim, Germany, 2011, pages 1-481. Non-limiting exemplary schemes for assembling the dual prodrug compounds are depicted in FIG. 9.

The use of the consolidated and dual prodrug compounds to treat cancers, such as pancreatic cancer, are described for specific example purposes. It should be understood, however, that other compounds can be useful for treating a variety of metabolic disorders due to their distinct interactions with well established cellular pathways, and especially with that of the PDX1-BIRC5-Survivin axis (which is likewise disclosed herein as specific examples). Certain embodiments of the compounds described herein are also useful for indications such as diabetes and metabolic syndrome. The compounds may also be administered with a small amount of digoxin to inhibit Na/K ATPase and further enhance their therapeutic properties. Similarly, co-administration according to common clinical practice with standard chemotherapeutic agents, particularly those that prompt apoptosis, are likewise beneficial for the cancer indications such as pancreatic cancer.

### EXAMPLES

Biological studies directed toward defining dosing regimens were conducted and showed that Met and Sim can be administered in a similar micromolar dose range, with latitude for Met to also be given at higher levels. Surprisingly, it was determined from cancer cell specimen studies that the use of Met and Sim is often just as effective as the administration of all three of the C3 drugs. This finding combined with the dose-equivalency shows that a consolidated or dual prodrug compound of Met−Sim may advantageously provide the same, if not better, therapeutic effect as the C3 combination. It was further found that various degrees of methylation on the Met system did not cause significant changes in whatever role metformin plays in the C3 mix, which indicates that chemical linkages can be added to one or more ends of Met during assembly of larger compounds.

Consolidated compounds composed of Met−Sim constructs were synthesized and tested, revealing that they maintained the desired activity normally present across both of the independent species when co-administered, and in some cases are synergistic compared to co-administration of the individual species.

### General Synthetic Chemistry Methods

Reactions were conducted in glass vessels that were cleaned with CH₃OH and acetone, and then dried in an oven. Reactions performed in round-bottom flasks were equipped with Teflon-coated magnetic stirrers. Solvents were removed under reduced pressure with gentle heating while using a Heidolph rotary evaporator (Hei-VAP Value, “The Collegiate”) connected to either a water aspirator or a diaphragm-driven vacuum pump. Reaction products were dried under high vacuum for 12 hrs at room temperature (RT). TLC was performed on Baker-flex TLC plates containing a fluorescent indicator (2.5×7.5 cm) and compounds were visualized by examination under short wave-length (254 nm) UV light. Melting points were determined for solid products on a MEL-TEMP II Laboratory Device and are uncorrected. NMR spectra were recorded on a Brucker Avanche-600 spectrophotometer (600 MHz). ¹H-NMR and ¹³C NMR were obtained in CDCl₃. Chemical shifts are reported in ppm (δ) and were referenced to the residual proton signal of the deuterated solvent. Chemical shifts for both ¹H-NMR and ¹³C-NMR are recorded to the second decimal. Peak multiplicities are abbreviated as singlet by s, doublet by d, triplet by t, quartet by q, and multiplet by m. Coupling constants are reported in hertz (Hz). Elemental analyses were performed for C, H, and N by Atlantic Microlab, Inc. with experimental results deemed acceptable for structural confirmation and sample purity when within +/−0.4% of theory.

### Synthesis of SAR Probes

SAR probes were synthesized as depicted in FIG. 5.

### Simvastatin-NHOH

Simvastatin (100 mg, 0.24 mmol) was dissolved in 0.5 mL of THF. 73 μL of 50% aq. NH₂OH (1.18 mmol) was added. The mixture was stirred at RT for 12 hrs and the THF evaporated. The residue was dissolved in 20 mL of DCM and washed twice with 20 mL of aq. HCl (pH 4-5). The separated DCM layer was dried over anhydrous sodium sulfate, concentrated by evaporation, and transferred onto a silica gravity column. The column was eluted with a step-gradient of 0 and then 6% MeOH/DCM. Fractions were assessed by TLC and those having product were combined using ethyl acetate to rinse each tube. Evaporation produced 50 mg (38%) of an oily residue. Rf: 0.3 (5% MeOH/DCM). ¹H-NMR (600 MHz, CDCl₃): δ 10.33 (s, 1H), 5.95 (d, J=9.6 Hz, 1H), 5.76 (dd, J=9.7, 5.7 Hz, 1H), 5.47 (t, J=3.1 Hz, 1H), 5.35 (d, J=4.3 Hz, 1H), 3.73 (s, 1H), 2.41-2.22 (m, 5H), 1.92 (d, J=13.56 Hz, 2H), 1.62-1.48 (m, 6H), 1.37 (m, 1H), 1.25 (m, 1H), 1.10 (d, J=3.66 Hz, 6H), 1.06 (d, J=7.32 Hz, 3H), 0.86 (d, J=6.7 Hz, 3H), 0.80 (t, J=7.5 Hz, 3H). ¹³C-NMR (151 MHz, CDCl₃): δ 178.48, 169.76, 133.28, 131.74, 129.33, 128.23, 71.57, 68.50, 68.43, 43.08, 43.04, 40.68, 37.48, 36.64, 34.98, 33.04, 32.83, 30.63, 27.27, 24.77, 24.73, 24.47, 23.05, 13.89, 9.36. Elemental analysis for C₂₅H₄₁NO₆·0.7 mole EtOAc: Theory C 65.05; H 9.15; N 2.73; Found C 64.76; H 8.96; N 3.00.

### Lovastatin-NHOH

Lovastatin (1 g, 2.47 mmol) was dissolved in 5 mL of THF. 750 μL of 50% aq. NH₂OH (5 eq., 12.25 mmol) was added. The mixture was stirred at RT for 10 hrs and the THF evaporated. The residue was dissolved in a minimal amount of anhydrous MeOH with gentle warming, and then water was added dropwise until the solution became cloudy. After the solution was chilled over an ice bath, pure solid product was obtained without the need for additional column purification. The supernatant was decanted and the remaining solid collected and dried to produce 750 mg (70%) of white, sticky amorphous solid. Rf: 0.5 (5% MeOH/DCM). ¹H-NMR (600 MHz, CDCl₃): δ 5.99 (d, J=9.6 Hz, 1H), 5.79 (dd, J=9.6, 6.0 Hz, 1H), 5.52 (dd, J=4.2, 2.2 Hz, 1H), 5.41 (q, J=3 Hz, 1H), 4.27 (tt, J=8.5, 4.0 Hz, 1H), 3.79-3.71 (m, 1H), 2.4-2.26 (m, 6H), 1.95-1.92 (m, 2H), 1.71-1.56 (m, 5H), 1.47-1.41 (m, 2H), 1.31-1.29 (m, 1H), 1.21-1.06 (m, 7H), 0.93-0.86 (m, 6H). ¹³C-NMR (151 MHz, CDCl₃): δ 177.59, 133.48, 131.81, 129.34, 128.19, 71.43, 68.32, 41.61, 37.34, 36.66, 34.96, 32.76, 30.67, 27.46, 26.83, 24.43, 22.90, 16.33, 13.90, 11.73. Elemental analysis for C₂₄H₃₉NO₆: Theory C 61.30; H 8.40; N 2.92; Found C 61.13; H 8.45, N 2.81.

### Synthesis of Consolidated Compounds (1:1 Met)

Consolidated compounds were synthesized as shown in the scheme depicted in FIG. 6.

Sim−Met (from Lactone)

Simvastatin (100 mg, 0.24 mmol) and metformin (70 mg, ca. 2 eq., 0.4 mmol) were dissolved in THF (5 ml) and the mixture was stirred at RT with reaction progress monitored by TLC. After completion at ca. 12 hrs, the THF was evaporated. The residue was dissolved in 20 mL DCM and washed twice with 10 mL of aq. HCl (pH 4-5) to remove excess metformin. The DCM layers in the separatory funnel were cloudy, but adding a few drops of methanol clarifies this and provides a sharper interface. The separated DCM layer was dried over anhydrous sodium sulfate and evaporated. The residue was crystallized from anhydrous methanol and water similar to that described above for lovastatin-NHOH. A distinctly crystalline product was obtained initially. This was collected by vacuum filtration, and the cloudy mother liquor was allowed to continue standing at RT for 48 hrs. The supernatant was decanted and the precipitate was collected and dried. Both crystals and precipitate had acceptable analytical specs. Combined yield of white solids was 45 mg (34%) with identical Rf=0.4 (5% MeOH/CH₂Cl₂) and melting points: 72-75° C. NMR data was appropriate for both solids and each entrained a small trace of DCM. Additional analytical data for each solid is provided below.

Crystalline Sim−Met (from lactone). 20 mg of white solid. ¹H-NMR (600 MHz, CDCl₃): δ 5.98 (dd, J=9.8, 2.2 Hz, 1H), 5.78 (dd, J=9.6, 6.1 Hz, 1H), 5.49 (t, J=3.1 Hz, 1H), 5.38-5.26 (m, 2H), 4.33-4.22 (m, 1H), 3.81 (dddd, J=9.5, 7.6, 5.4, 2.7 Hz, 1H), 3.14-3.08 (m, 6H), 2.70-2.58 (m, 2H), 2.47-2.35 (m, 2H), 2.24 (dq, J=11.9, 2.7 Hz, 1H), 2.01-1.89 (m,2H), 1.69-1.49 (m, 6H), 1.44-1.40 (m, 1H), 1.28-1.22 (m, 1H), 1.12 (d, J=5.5 Hz, 6H), 1.07 (d, J=7.44 Hz, 3H), 0.88 (dd, J=7.1, 2.3Hz, 3H), 0.82 (td, J=7.5, 2.2 Hz, 3H). ¹³C-NMR (151 MHz, CDCl₃): δ 177.83, 175.95, 175.93, 165.90, 164.46, 133.38, 131.87, 129.40, 128.26, 72.63, 70.62, 68.10, 44.10, 42.95, 42.73, 37.53, 36.48, 36.21, 34.83, 33.01, 32.77, 30.64, 27.32, 24.76, 24.70, 24.55, 23.04, 13.85, 9.32. Elemental analysis for C₂₉H₄₉N₅O₅·0.2 mole DCM: Theory C 62.10; H 8.82; N 12.40; Found: C 62.44; H 8.47; N 12.50.

Precipitated Sim−Met (from lactone). 25 mg of white solid. ¹H-NMR (600 MHz, CDCl₃): δ 5.99 (d, J=9.6 Hz, 1H), 5.79 (dd, J=9.6, 6.1 Hz, 1H), 5.50 (t, J=3.1 Hz, 1H), 5.37 (q, J=3.2 Hz, 1H), 5.19 (s, 2H), 4.27 (dtd, J=8.6, 6.1, 3.4 Hz, 1H), 3.83 (dddd, J=9.8, 8.0, 5.6, 2.7 Hz, 1H), 3.12 (q, J=7.6, 5.4 Hz, 6H), 2.71-2.59 (m, 2H), 2.47-2.36 (m, 2H), 2.25-(dq, J=12.2, 2.7 Hz, 1H), 2.03-1.90 (m, 2H), 1.73-1.49 (m, 6H), 1.44 (tt, J=12.8, 3.9 Hz, 1H), 1.33-1.22 (m, 1H), 1.11 (d, J=5.58 Hz, 6H), 1.08 (d, J=7.74 Hz, 3H), 0.90 (d, J=6.96 Hz, 3H), 0.84 (t, J=7.4 Hz, 3H). ¹³C-NMR (151 MHz, CDCl₃): δ 177.84, 175.98, 165.85, 164.46, 133.40, 131.89, 129.42, 128.26, 76.68, 72.68, 70.65, 68.12, 44.07, 42.96, 42.71, 37.54, 36.74, 36.50, 36.22, 34.83, 33.02, 32.78, 30.65, 27.33, 24.75, 24.71, 24.55, 23.04, 13.85, 9.33. Elemental analysis for C₂₉H₄₉N₅O₅·0.3 mole of DCM: Theory C 61.39; H 8.72; N 12.24; Found: C 61.07; H 8.35; N 12.24.

Lov−Met (from Lactone)

This reaction was conducted in an identical manner except that lovastatin was substituted for simvastatin. All of the product was collected as the precipitated material rather than initially isolating a crystalline form.

### Sim/Lov−Met (at the Statins' Ester Site)

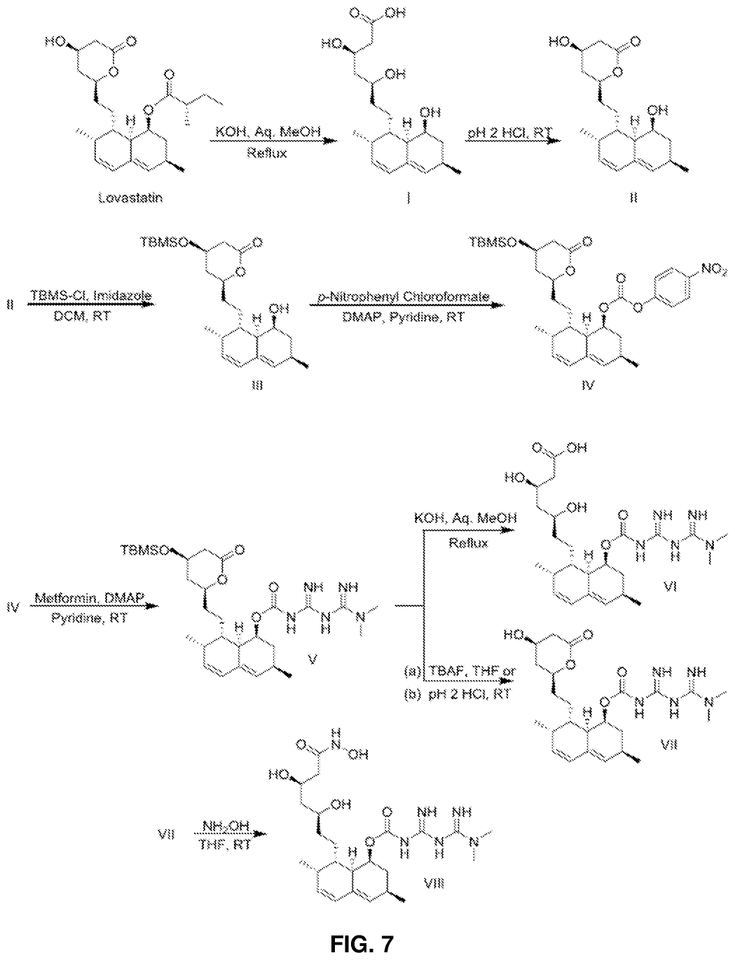

Sim−Met and Lov−Met were synthesized at the statins' ester site as depicted in FIG. 7.

### TBMS-Protected Des-ester Sim/Lov (Intermediate III)

Lovastatin (1 g, 2.47 mmol) was heated with 1.4 g KOH (10 eq., 24.78 mmol) in 30 mL H₂O/MeOH (⅙) at reflux for 3 days. MeOH was removed under reduced pressure. The resulting des-ester, opened-lactone intermediate I was used directly in the next step by adding 40 mL of water and 30 mL of DCM to the residue, and adjusting the pH to 2-3 with conc. HCl. The mixture was stirred at RT for 12 hrs., neutralized with saturated aq. NaHCO₃, and extracted with DCM (2×30 mL). The combined organic phases were evaporated and the crude intermediate II having a closed lactone was used immediately in the next step by treatment with tert-butyldimethylsilyl chloride (TBMSCI) (0.97 g, 6.43 mmol) and imidazole (0.98 g, 14.34 mmol) in 40 mL DCM at RT for 5 hrs. The mixture was concentrated under reduced pressure, the residue extracted with 40 mL of DCM and the extract washed twice with 20 mL of water. The organic phase was dried over Na₂SO₄, filtered, concentrated, and purified by gravity chromatography (silica gel using a step-gradient of EtOAc/hexane ( 2/8) to EtOAc). Fractions were assessed by TLC and those having product were combined and evaporated to provide 350 mg (33% overall) of white solid: Mp 135-138° C.; Rf 0.6 (EtOAc/Hex 20:80); ¹H-NMR (600 MHz, CDCl₃): δ 5.85 (d, J=9.6 Hz, 1H), 5.65 (dd, J=9.5, 6.1 Hz, 1H), 5.40 (t, J=3.3 Hz, 1H), 4.57 (ddt, J=11.3, 6.9, 3.4 Hz, 1H), 4.19 (m, 1H), 4.12 (q, J=3.0 Hz, 1H), 2.51 (dd, J=17.4, 4.4 Hz, 1H), 2.42 (ddd, J=17.4, 3.3, 1.9 Hz, 1H), 2.28 (dtd, J=35.6, 7.2, 4.4 Hz, 1H), 2.03 (dq, J=12.2, 2.7 Hz, 1H), 1.80-1.57 (m, 7H), 1.45-1.28 (m,2H), 1.13 (t, J=7.2 Hz, 4H), 1.07 (d, J=7.5 Hz, 3H), 0.78 (d, J=9.1 Hz, 12H). ¹³C-NMR (151 MHz, CDCl₃): δ 170.31, 133.26, 131.37, 129.84, 128.54, 76.21, 64.80, 63.56, 39.18, 38.66, 36.67, 36.19, 35.75, 32.86, 30.72, 27.46, 25.68, 25.64, 25.60, 24.01, 23.55, 20.85, 17.84, 13.88, −5.01, −5.19.

### TBMS-Protected SIM/LOV Carbonate (Intermediate IV)

Intermediate III (100 mg, 0.23 mmol), p-nitrophenyl chloroformate (0.324 g, 1.61 mmol), and DMAP (0.2 g, 1.61 mmol) were mixed in anhydrous pyridine (16 mL) and stirred at RT for 15 hrs. Pyridine was removed under reduced pressure, and the residue was dissolved in 20 mL of DCM and washed with 10 mL of 1N HCl, 10 mL of saturated NaHCO₃ solution, and 10 mL of brine. The separated organic layer was dried over Na₂SO₄, filtered, and evaporated. A TLC (Rf 0.3 20% EtOAc/Hex) and NMR of the crude product were appropriate so the material was used directly in the next step without additional purification.

TBMS-Protected SIM/LOV−MET (from Ester) (Penultimate Intermediate V)

Carbonate IV (70 mg, 0.12 mmol), metformin (0.11 g, 7 eq., 0.845 mmol), and DMAP (0.1 g, 7 eq., 0.845 mmol) were dissolved in 5 mL of pyridine. The reaction was stirred at RT for 20 hrs and then evaporated. The residue was dissolved in 20 mL of DCM and washed with 10 mL of 1N HCl, 10 mL of sat. NaHCO₃, and 10 mL of brine. The separated organic layer was dried over Na₂SO₄, filtered, and evaporated. A TLC and NMR of the crude product were appropriate so the material was used directly in the final step without additional purification.

SIM/LOV−MET (from Ester)—Versions VI and VII

Removal of the TBMS-protecting group using strongly basic conditions can also open the lactone ring (VI), whereas removal by either acidic aqueous conditions (pH ca. 2) or by using tetrabutylammonium fluoride (TBAF) in anhydrous THF can retain the closed lactone ring (VII).

SIM/LOV−MET (from Ester)—Version VIII

Reaction of VII under conditions similar to those used to produce SAR probes simvastatin-NHOH and lovastatin-NHOH can be used to prepare the analogous ring-opened analog of the SIM/LOV−NHOH−MET (from ester) version VIII.

Synthesis of Consolidated Compound with a Ratio of one Statin to Two Mets

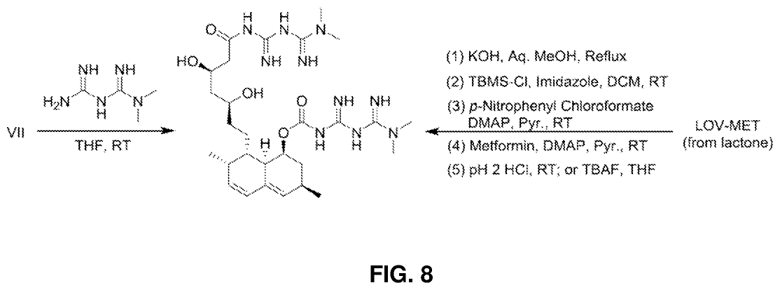

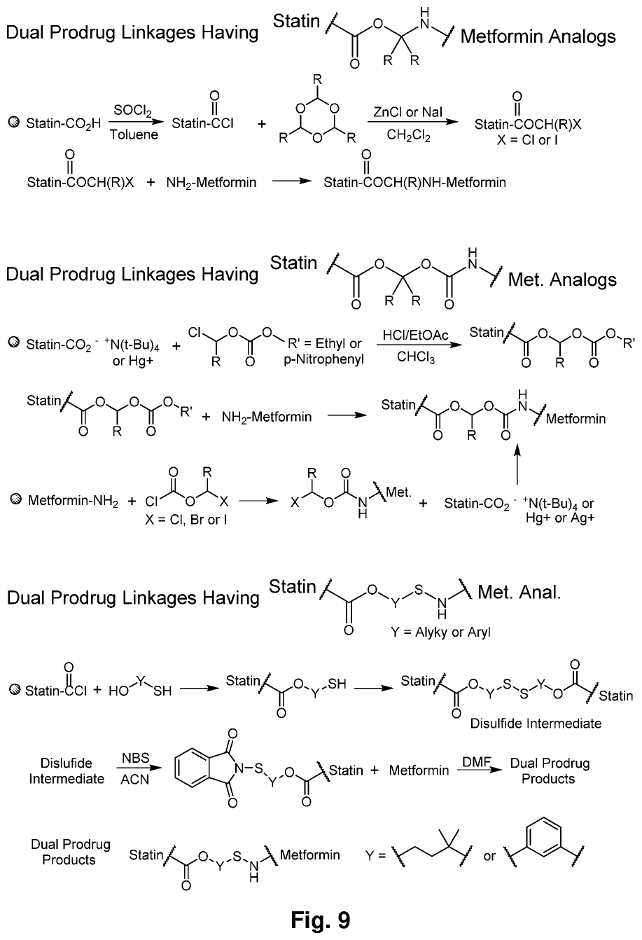

A consolidated compound having a Met:statin molar ratio of 2:1 may be synthesized as depicted in FIG. 8.

The statin-MET (1:2 MET) analog depicted in FIG. 8 can be prepared by adding a second Met to compound VII using the chemistry specified for synthesis of the Sim−Met or Lov−Met compounds. Alternatively, the Sim−Met or Lov−Met compounds can be deployed as the starting materials to follow the 5-step pathway specified in FIG. 8, which is similar to how VII was produced except that re-closure of the lactone is no longer appropriate and two TBMS moieties are needed to protect both of the alkyl-chain hydroxyl-groups as a prelude to steps 3 and 4. Final removal of the TBMS protecting groups can again be done by either of 2 methods.

### Synthesis of Dual Prodrug Compounds

Commonly used chemistry from the single prodrugs arena can likewise be used for assembly of the dual prodrugs. Several exemplary routes are depicted in FIG. 9 which are not meant to be inclusive.

### General Biology Testing Methods

The methods used to test anticancer activity have been previously described. The method used to assess alteration of metabolic activity deploys some of the same cell lines that have previously been used for the anticancer activity but instead focuses directly upon measuring ATP levels using commercially available assay kits. This example shows a direct comparison of the consolidated compounds' activities versus the actions of Sim and Met when administered simultaneously as separate agents.

### Anticancer Activity

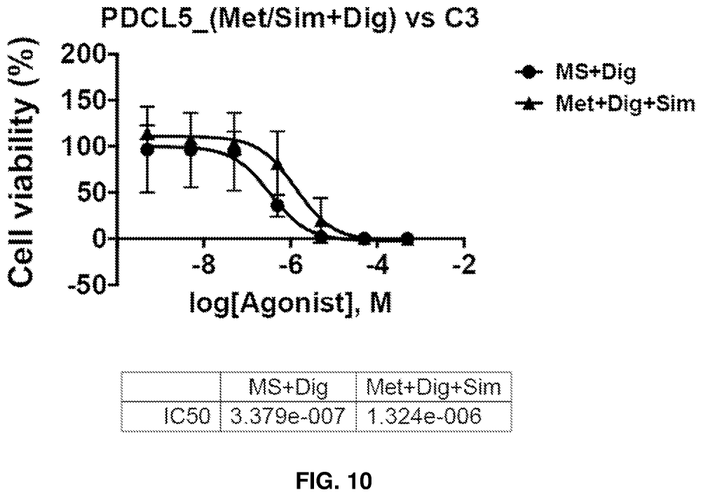

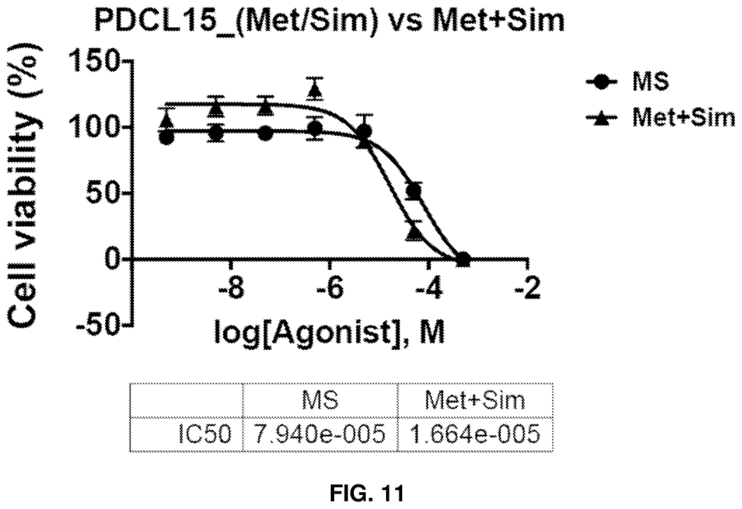

The results of anticancer activity assays are shown in FIGS. 10-11. In most of the cancer cell lines, the action of the consolidated compound Sim−Met (from lactone) had comparable activity to when Sim+Met were given simultaneously as independent compounds, with or without also adding digitalis (DIG) in both protocols. For the particular cancer cell line of PDCL5, however, our consolidated Sim−Met construct was surprisingly more potent toward inhibiting cancer cell growth (note the lower IC₅₀ value).

As recited in FIGS. 10-11, “SIM−MET (from lactone)” is either “Met/Sim” as in the title or is “MS” as in the graph legend and in the tabulated IC₅₀ values; whereas the independent agents are either “C3” as in the title or are “Met+Dig+Sim.” In this first case, both administration protocols included digitalis (“Dig”). Continuing assessments of patient cancer biopsy/cell cultures has indicated that the Dig component may not always be additionally beneficial. Thus, the Sim−Met combination may be ideal for personalizing cancer therapy so as to be suited for use with Dig also added or with Dig not present based upon individual biopsy optimization. An example of just a two component (no Dig present) comparison is present in FIG. 11. In this case the patient's personalized cancer cell (“PDCL15”) response is comparable between the consolidated compound (“MS”) and the independent simultaneous drug administration (“Met+Sim”) protocols.

### Metabolic Activity

FIG. 12 demonstrates the ability of Met−Sim to impact upon ATP levels in a manner comparable to when Met and Sim are mixed together and simultaneously administered. FIG. 13 shows another example of comparable activity at the cell culture level while deploying a different cell type (“Mia PaCa2”), which are human pancreatic cancer cells.

Certain embodiments of the compositions, compounds, and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions, compounds, and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A composition comprising formula A: 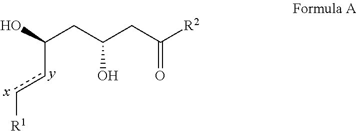
wherein R¹ is one of the following statin multi-cyclic core moieties R^{1a-f} 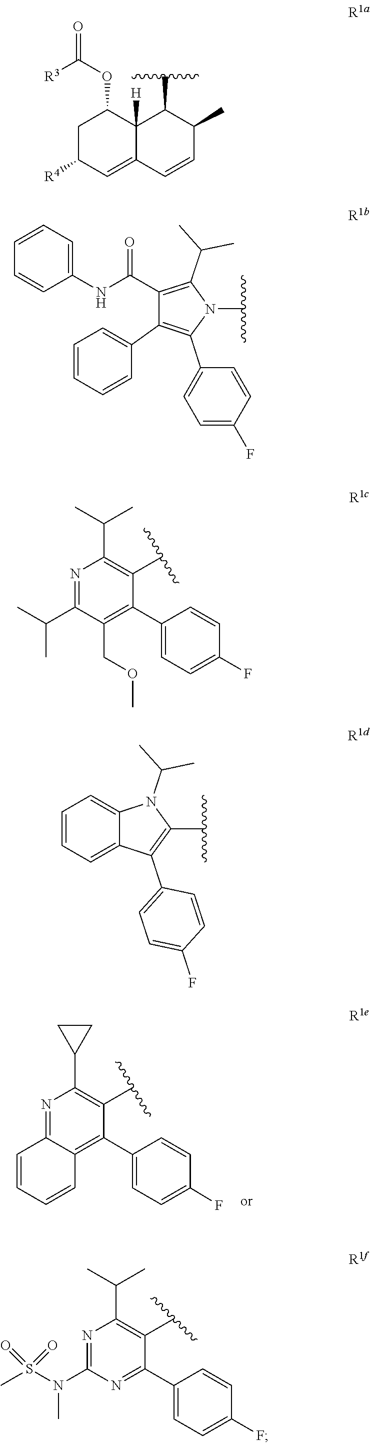
the x-y bond is single or double and has an (E)-conformation;
R² is 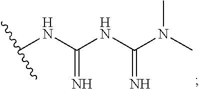 [SMILES: *NC(=N)NC(=N)N(C)C]
R³ is CH₂CH₂CH₃, CH(CH₃)CH₂CH₃ or C(CH₃)₂CH₂CH₃; and
R⁴ is H, CH₃, OCH₃ or OH;
and enantiomers, racemates, diastereomers, hydrates, and solvates thereof.

2-3. (canceled)

4. The composition of claim 1, having the one of the following structures:

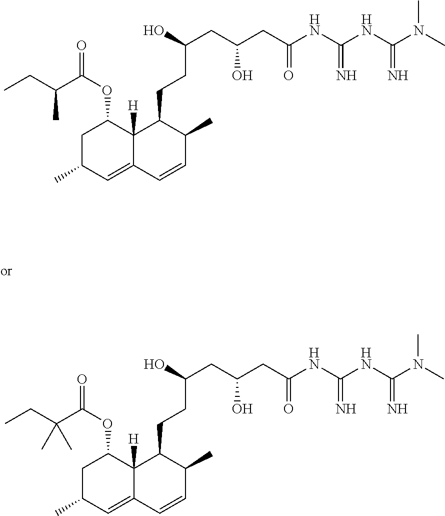

5-9. (canceled)

10. A composition comprising one of the following formulas B: 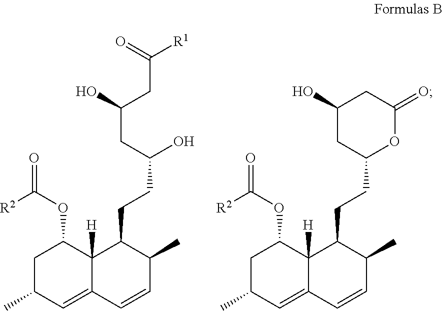
wherein R¹ is OH, OMe, OEt, NHOH, or 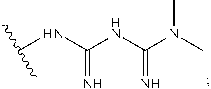 [SMILES: *NC(=N)NC(=N)N(C)C]
R² is 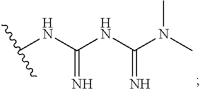 [SMILES: *NC(=N)NC(=N)N(C)C]
and enantiomers, racemates, diastereomers, hydrates, and solvates thereof.

11-16. (canceled)

17. A method of treating cancer or a metabolic disorder, the method comprising administering an effective amount of a compound of formulas A, B, or D to a subject in need of such therapy: 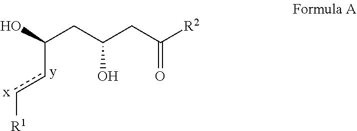
wherein R¹ is one of the following statin multi-cyclic core moieties R^{1a-f} 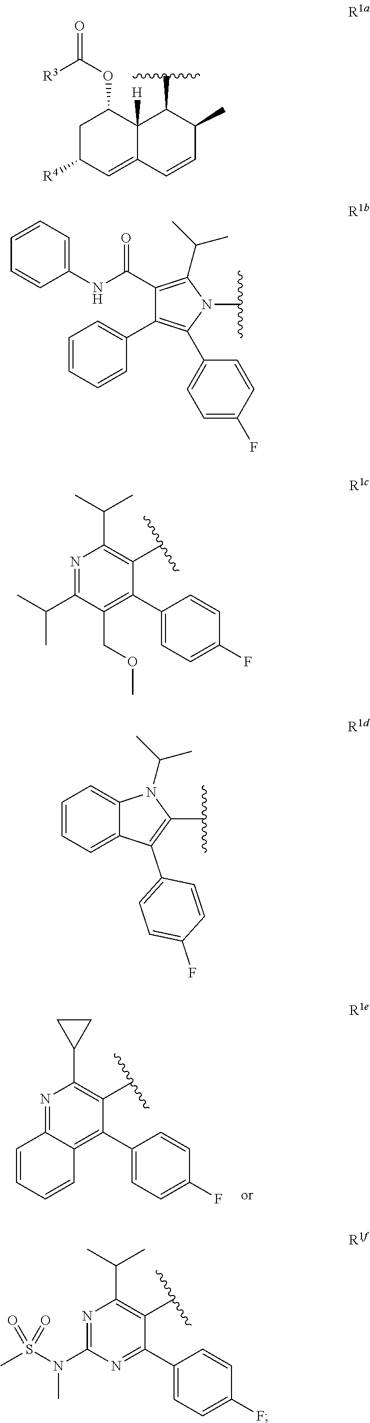
the x-y bond is single or double and has an (E)-conformation;
R² is 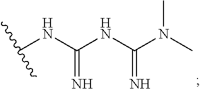 [SMILES: *NC(=N)NC(=N)N(C)C]
R³ is CH₂CH₂CH₃, CH(CH₃)CH₂CH₃, or C(CH₃)₂CH₂CH₃;
R⁴ is H, CH₃, OCH₃, or OH; and
and enantiomers, racemates, diastereomers, hydrates, and solvates thereof; or 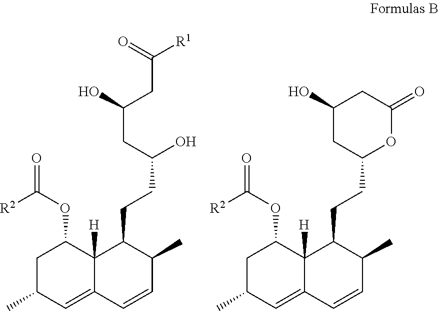
wherein R¹ is OH, OMe, OEt, NHOH, or 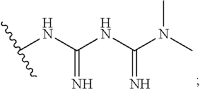 [SMILES: *NC(=N)NC(=N)N(C)C]
and
R² is 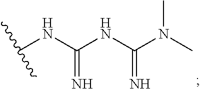 [SMILES: *NC(=N)NC(=N)N(C)C]
and enantiomers, racemates, diastereomers, hydrates, and solvates thereof; or 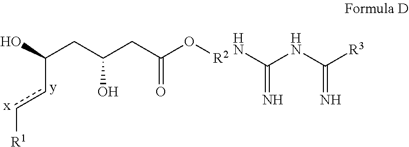
wherein R¹ is one of the following statin multi-cyclic core moieties R1^{a-f} 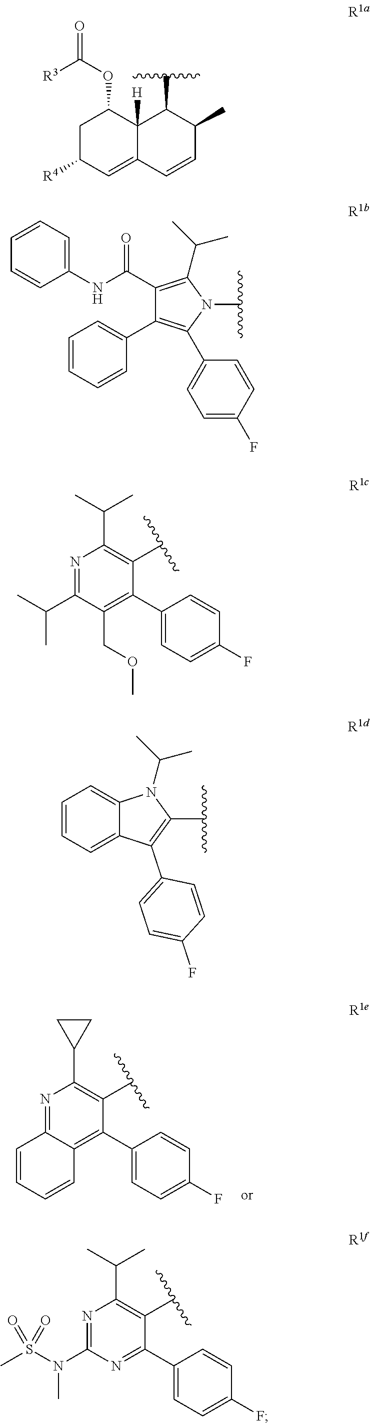
the x-y bond is single or double and has an (E)-conformation;
R² is CH₂, CH(CH₃), CH(CH₂CH₃), CH(CH₂CH₂CH₃), CH[CH(CH₃)₂], C(CH₃)₂, C(CH₂CH₃)₂, CH₂O(CO), CH(CH₃)O(CO), CH(CH₂CH₃)O(CO), CH(CH₂CH₂CH₃)O(CO), CH[CH(CH₃)₂]O(CO), C(CH₃)₂O(CO), C(CH₂CH₃)₂O(CO), CH₂CH₂C(CH₃)₂S, or 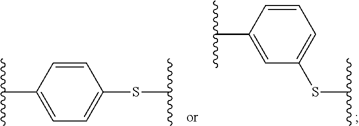 [SMILES: *Sc1ccc(*)cc1 ; *Sc1cccc(*)c1]
R³ is 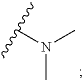 [SMILES: *N(C)C]
R⁴ is CH₂CH₂CH₃, CH(CH₃)CH₂CH₃, or C(CH₃)₂CH₂CH₃;
R⁵ is H, CH₃, OCH₃, or OH;
and enantiomers, racemates, diastereomers, hydrates, and solvates thereof.

18-25. (canceled)

26. The method of claim 17, wherein the cancer is pancreatic cancer or a cancer of the GI tract, including each of their metastatic locales.

27. The method of claim 17, further comprising administering a cardiac glycoside or one or more chemotherapeutic agents, or both a cardiac glycoside and one or more chemotherapeutic agents, with the compound of formulas A, B, or D, wherein the one or more chemotherapeutic agents by pharmacologic characterization at least partially relies upon prompting apoptosis to kill cancer cells.

28-33. (canceled)

34. The method of claim 26, further comprising administering a combination of four drugs called folfirinox with the compound of formulas A, B, or D, wherein the folfirinox comprises leucovorin, 5-fluorouracil, irinotecan, and oxaliplatin.

35. A composition comprising formula D: 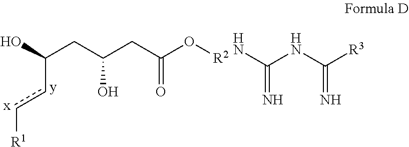
wherein R¹ is one of the following statin multi-cyclic core moieties R^{1a-f} 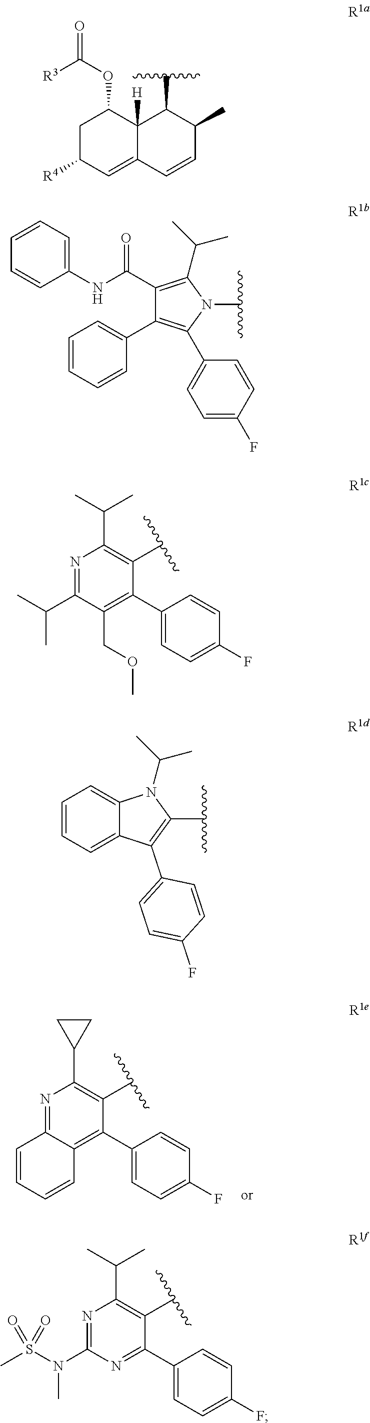
the x-y bond is single or double and has an (E)-conformation;
R² is CH₂, CH(CH₃), CH(CH₂CH₃), CH(CH₂CH₂CH₃), CH[CH(CH₃)₂], C(CH₃)₂, C(CH₂CH₃)₂, CH₂O(CO), CH(CH₃)O(CO), CH(CH₂CH₃)O(CO), CH(CH₂CH₂CH₃)O(CO), CH[CH(CH₃)₂]O(CO), C(CH₃)₂O(CO), C(CH₂CH₃)₂O(CO), CH₂CH₂C(CH₃)₂S, or 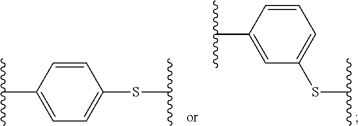 [SMILES: *Sc1ccc(*)cc1 ; *Sc1cccc(*)c1]
R³ is 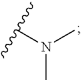 [SMILES: *N(C)C]
R⁴ is CH₂CH₂CH₃, CH(CH₃)CH₂CH₃, or C(CH₃)₂CH₂CH₃;
R⁵ is H, CH₃, OCH₃ or OH;
and enantiomers, racemates, diastereomers, hydrates, and solvates thereof.

36. The composition of claim 35, wherein R¹ is R^{1a}, the x-y bond is single, R⁴ is CH(CH₃)CH₂CH₃ or C(CH₃)₂CH₂CH₃, and R⁵ is CH₃.

37. The composition of claim 36, wherein R² is CH(CH₃) or C(CH₃)₂.

38. The composition of claim 36, wherein R² is CH(CH₃)O(CO) or C(CH₃)₂O(CO).

39. The composition of claim 36, wherein R² is CH₂CH₂C(CH₃)₂S.

40-53. (canceled)

54. The method of claim 17, wherein the metabolic disorder is one or more of the following: a glucose metabolism disorder, hyperlactatemia, a lipid metabolism disorder, or a phosphorous metabolism disorder.

55. The method of claim 54, wherein the metabolic disorder is type 2 diabetes or is associated with hyperlipidemia or metabolic syndrome.

56. The method of claim 17, wherein the cancer's overall pharmacologic characterization includes involvement of the BIRC5-Survivin Axis.

57. A method of treating hyperlipidemia or metabolic syndrome, the method comprising administering an effective amount of a compound having formula A to a human subject in need of such therapy: 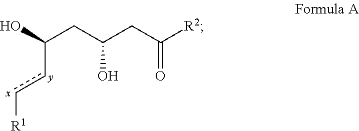
wherein R¹ is the following statin multi-cyclic core moiety R^{1a} 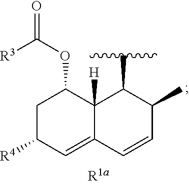
the x-y bond is a single bond having an (E)-conformation;
R² is 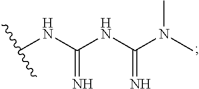 [SMILES: *NC(=N)NC(=N)N(C)C]
R³ is CH(CH₃)CH₂CH₃, C(CH₃)₂CH₂CH₃, or 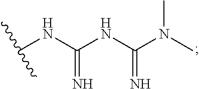 [SMILES: *NC(=N)NC(=N)N(C)C]
and
R⁴ is CH₃.

58. A method of treating hyperlipidemia or metabolic syndrome, the method comprising administering an effective amount of a compound having formula D to a human subject in need of such therapy: 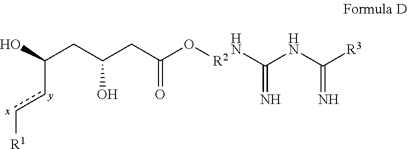
wherein:
the x-y bond is a single bond;
R¹ is the following statin multi-cyclic core moiety R^{1a}:
R² is CCH₃R⁶; 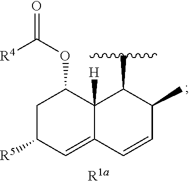
R³ is 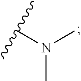 [SMILES: *N(C)C]
R⁴ is CCH₃R⁶CH₂CH₃;
R⁵ is CH₃; and
each R⁶ is, independently, H or CH₃.

59. A method of treating pancreatic or GI-tract cancers, the method comprising administering an effective amount of a compound having formulas A or D to a human subject in need of such therapy: 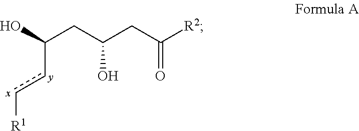
wherein:
R¹ is the following statin multi-cyclic core moiety R^{1a} 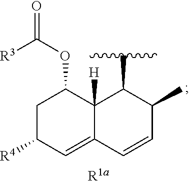
the x-y bond is a single bond;
R² is 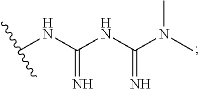 [SMILES: *NC(=N)NC(=N)N(C)C]
R³ is CH(CH₃)CH₂CH₃, C(CH₃)₂CH₂CH₃, or 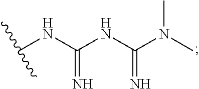 [SMILES: *NC(=N)NC(=N)N(C)C]
and
R⁴ is CH₃;
or 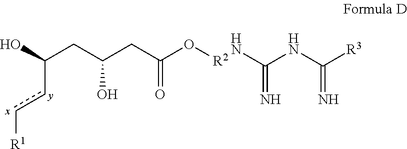
wherein:
the x-y bond is a single bond having an (E)-conformation;
R¹ is the following statin multi-cyclic core moiety R^{1a}:
R² is CCH₃R⁶; 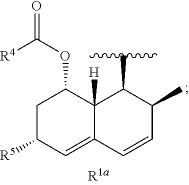
R³ is 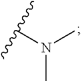 [SMILES: *N(C)C]
R⁴ is CCH₃R⁶CH₂CH₃;
R⁵ is CH₃; and
each R⁶ is, independently, H or CH₃.

60. The method of claim 59, further comprising administering a cardiac glycoside, or one or more chemotherapeutic agents, or both a cardiac glycoside and one or more chemotherapeutic agents, with the formula of formulas A or D, wherein the one or more chemotherapeutic agents by pharmacologic characterization at least partially rely upon prompting apoptosis to kill cancer cells.

61. The method of claim 59, further comprising administering a combination of four drugs called folfirinox with the compound of formulas A, B, or D, wherein the folfirinox comprises leucovorin, 5-fluorouracil, irinotecan, and oxaliplatin.
